(19) United States
(12) Patent Application Publication
(10) Pub. No.: US 2002/0040046 A1
(43) Pub. Date: Apr. 4, 2002

(54) Novel formulations comprising lipid-regulating agents

(76) Inventors: Jitendra P Patel, Libertyville, IL (US); Yeshwant D Sanzgiri, Gurnee, IL (US); John M Lipari, Racine, WI (US); Thomas L Reinland, Gages Lake, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 09/524,113

(22) Filed: Mar. 13, 2000

Related U.S. Application Data
(60) Provisional application No. 60/127,136, filed on Mar. 31, 1999.

(51) Int. Cl. A61K 31/40; A61K 31/35; A61K 31/21

(52) U.S. Cl. 514/410; 514/460; 514/510

(57) ABSTRACT

The present invention is directed to a formulation comprising a lipid-regulating agent dissolved or dispersed in at least one oil and an emulsifier or emulsifier blend, the resulting mixture being capable of forming an emulsion upon dilution in an aqueous medium.

### REFERENCE TO RELATED APPLICATION

This application is a conversion of U.S. Provisional patent application 60/127,136, filed on Mar. 31, 1999.

### FIELD OF THE INVENTION

The present invention relates to novel formulations comprising lipid-regulating agents.

### BACKGROUND OF THE INVENTION

2-[4-(4-chlorobenzoyl)phenoxy]-2-methyl-propanoic acid, 1-methylethylester, also known as fenofibrate, is representative of a broad class of compounds having pharmaceutical utility as lipid regulating agents. More specifically, this compound is part of a lipid-regulating agent class of compounds commonly known as fibrates, and is disclosed in U.S. Pat. No. 4,058,552.

Fenofibrate has been prepared in several different formulations, c.f., U.S. Pat. No. 4,800,079 and U.S. Pat. No. 4,895,726. U.S. Pat. No. 4,895,726 discloses a co-micronized formulation of fenofibrate and a solid surfactant.

U.S. Pat. No. 4,961,890 discloses a process for preparing a controlled release formulation containing fenofibrate in an intermediate layer in the form of crystalline microparticles included within pores of an inert matrix. The formulation is prepared by a process involving the sequential steps of dampening said inert core with a solution based on said binder, then projecting said fenofibrate microparticles in a single layer onto said dampened core, and thereafter drying, before said solution based on said binder dissolves said fenofibrate microparticles, and repeating said three steps in sequence until said intermediate layer is formed.

European Patent Application No. EP0793958A2 discloses a process for producing a fenofibrate solid dosage form utilizing fenofibrate, a surface active agent and polyvinyl pyrrolidone in which the fenofibrate particles are mixed with a polyvinyl pyrrolidone solution. The thus obtained mixture is granulated with an aqueous solution of one or more surface active agents, and the granulate thus produced is dried.

PCT Publication No. WO 82/01649 discloses a fenofibrate formulation having granules that are comprised of a neutral core that is a mixture of saccharose and starch. The neutral core is covered with a first layer of fenofibrate, admixed with an excipient and with a second microporous outer layer of an edible polymer.

U.S. Pat. No. 5,645,856 describes the use of a carrier for hydrophobic drugs, including fenofibrate, and pharmaceutical compositions based thereon. The carrier comprises a digestible oil and a pharmaceutically-acceptable surfactant component for dispersing the oil in vivo upon administration of the carrier, which comprises a hydrophilic surfactant, said surfactant component being such as not to substantially inhibit the in vivo lipolysis of the digestible oil.

Gemfibrozil is another member of the fibrate class of lipid-regulating agents. U.S. Pat. No. 4,927,639 discloses a disintegratable formulation of gemfibrozil providing both immediate and sustained release, comprising a tablet compressed from a mixture of a first and second granulation, and a disintegration excipient operable to effect partial or complete disintegration in the stomach. The first granulation comprises finely divided particles of pure gemfibrozil granulated with at least one cellulose derivative, and the second granulation comprises finely divided particles of pure gemfibrozil granulated with a pharmaceutically-acceptable water soluble or insoluble polymer which are then uniformly coated with a pharmaceutically-acceptable (meth)acrylate copolymer prior to admixture with the first granulation. The first and second granulations are present in the final composition in a ratio of from about 10:1 to about 1:10.

U.S. Pat. No. 4,925,676 discloses a disintegratable gemfibrozil tablet providing both immediate and enteric release, which is compressed from a mixture of a first granulation of gemfibrozil with at least one acid-disintegratable binder, and a second granulation formed from the first granulation, but regranulated or coated with an alkali-disintegratable formulation of at least one substantially alkali-soluble and substantially acid-insoluble polymer.

Another class of lipid-regulating agents are commonly known as statins, of which pravastatin and atorvastatin are members. U.S. Pat. Nos. 5,030,447 and 5,180,589 describe stable pharmaceutical compositions, which when dispersed in water have a pH of at least 9, and include a medicament which is sensitive to a low pH environment, such as pravastatin, one or more fillers such as lactose and/or microcrystalline cellulose, one or more binders, such as microcrystalline cellulose (dry binder) or polyvinylpyrrolidone (wet binder), one or more disintegrating agents such as croscarmellose sodium, one or more lubricants such as magnesium stearate and one or more basifying agents such as magnesium oxide.

It is an object of the present invention to provide formulations of lipid-regulating agents having enhanced bioavailability and longer half-life when compared to commercially available formulations.

### SUMMARY OF THE INVENTION

The present invention is directed to a formulation comprising a lipid-regulating agent dissolved in an oil, with subsequent emulsification using one or more emulsifiers. This formulation forms fine and stable emulsions. The emulsions result in an increase in drug solubility, oral bioavailability and half-life.

The formulation may be administered directly, diluted into an appropriate vehicle for administration, encapsulated into soft or hard gelatin shells or capsules for administration, or administered by other means obvious to those skilled in the art.

### BRIEF DESCRIPTION OF THE DRAWINGS

FIG. 1 is a graph showing the plasma concentration in fasted dogs of the formulation of Example 1 and a reference compound.

### DETAILED DESCRIPTION OF THE INVENTION

The bulk lipid-regulating agent may be prepared by any available method, as for example the compound fenofibrate may be prepared by the procedure disclosed in U.S. Pat. No. 4,058,552, or the procedure disclosed in U.S. Pat. No. 4,739,101, both herein incorporated by reference.

The solution comprising the lipid-regulating agent is prepared by dissolving said agent in the oil with adequate mixing. An emulsifier or emulsifier blend is added to said mixture and mixed until uniform. If desired, water can be then added to the resulting mixture with agitation to form a uniform emulsion.

The delivery system of the present invention results in increased solubility, half-life and bioavailability of the lipid-regulating agent. It can be further diluted with additional liquids or it may be thickened and/or stabilized with various pharmaceutical excipients to vary its existing properties.

Suitable oils include, but are not limited to, any pharmaceutically acceptable oil, such as, for example, soybean oil, coconut oil, canola oil, corn oil, palm kernel oil, cottonseed oil, olive oil, peanut oil, safflower oil and sesame oil.

Suitable emulsifiers include any pharmaceutically acceptable hydrophilic or lipophilic emulsifier or combinations thereof, such as, for example, phospholipids, polyoxyethylene sorbitan fatty acid derivatives, sorbitan fatty acid derivatives, polyoxyl-35-castor oil (Cremophor EL, available from BASF), castor oil or hydrogenated castor oil ethoxylates, polyglycerol esters of fatty acids, fatty acid ethoxylates, alcohol ethoxylates, polyoxyethylenepolyoxypropylene co-polymers and block co-polymers, and TPGS (d-alpha tocopheryl polyethylene glycol 1000 succinate). Preferred emulsifiers include polyoxyethylene sorbitan fatty acid derivatives, sorbitan fatty acid derivatives and polyoxyl-35-castor oil (Cremophor EL, available from BASF).

Other optional ingredients which may be included in the compositions of the present invention are those which are conventionally used in oil-based drug delivery systems, e.g. antioxidants such as, for example, tocopherol, ascorbyl palmitate, ascorbic acid, butylated hydroxytoluene, butylated hydroxyanisole, propyl gallate, etc.; pH stabilizers such as, for example, citric acid, tartaric acid, fumaric acid, acetic acid, glycine, arginine, lysine, potassium hydrogen phosphate, etc.; thickeners/suspending agents such as, for example, hydrogenated vegetable oils, beeswax, colloidal silicon dioxide, gums, celluloses, silicates, bentonite, etc.; flavoring agents such as, for example, cherry, lemon, aniseed flavors, etc.; sweeteners such as, for example, aspartame, saccharin, cyclamates, etc.; and co-solvents, such as, for example, ethanol, propylene glycol, polyethylene glycol, dimethyl isosorbide, etc.

The resulting liquid comprising the lipid-regulating agent may be dosed directly for oral administration, diluted into an appropriate vehicle for oral administration, filled into soft or hard shells or capsules for oral administration, or delivered by some other means obvious to those skilled in the art. The said liquid can be used to improve the oral bioavailability, and increase the half-life and solubility of said lipid-regulating agent.

The invention will be understood more clearly from the following non-limiting representative examples:

### Example 1

SR Soybean oil (24.33 g) was added to a beaker and fenofibrate (0.67 g) was dissolved in it by stirring. Sorbitan monooleate (2.5 g) was added to the beaker and mixed until uniform. Polysorbate 80 (0.5 g) was then added and mixed until uniform. Finally water (72 g) was added slowly with constant mixing until a uniform emulsion resulted.

### Example 2

SR Soybean oil (24 g) is added to a beaker and pravastatin (1 g) is dispersed in it by stirring. Sorbitan monooleate (2.5 g) is added to the beaker and mixed until uniform. Polysorbate 80 (0.5 g) is then added and mixed until uniform. Finally water (7.2 g) is added slowly with constant mixing until a uniform emulsion resulted.

### Example 3

SR Soybean oil (24 g) is added to a beaker and atorvastatin (1 g) is dispersed in it by stirring. Sorbitan monooleate (2.5 g) is added to the beaker and mixed until uniform. Polysorbate 80 (0.5 g) is then added and mixed until uniform. Finally water (7.2 g) is added slowly with constant mixing until a uniform emulsion resulted.

### Example 4

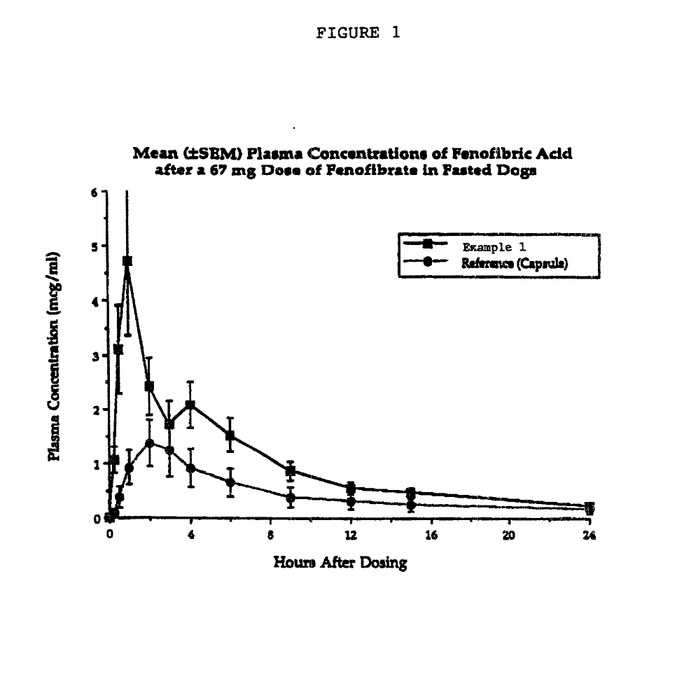

The emulsion prepared by the process described in Example 1, and from a commercial fenofibrate composition, Lipanthyl 67M (Groupe Fournier) (Reference), were administered to a group of dogs at a dose of 67 mg fenofibrate/dog (10 mL emulsion or one capsule/dog). The plasma concentrations of fenofibric acid were determined by HPLC. Concentrations were normalized to a 6.7 mg/kg dose in each dog. FIG. 1 presents the resulting data in graph form. The results provided as mean ±SD, n=6, were as follows:

Lipanthyl 67M (Reference):

Cmax=1.88±0.97 mcg/ml

Tmax=1.6±0.9 hr

t_{½}=4.5 hr

AUC (0-24)=11.08±9.42 mcg•hr/ml

Emulsion of Example 1:

Cmax=4.97±3.13 mcg/ml

Tmax=1.1±0.5 hr

t_{½}=7.8 hr

AUC (0-24)=24.21±11.69 mcg•hr/ml

AUC relative to Reference=2.2

1. A composition comprising a lipid-regulating agent dissolved or dispersed in at least one oil with one or more emulsifiers, wherein the mixture is capable of forming an emulsion upon dilution with an aqueous phase.

2. A composition of claim 1 wherein said lipid-regulating agent is a fibrate.

3. A composition of claim 2 wherein said fibrate is fenofibrate.

4. A composition of claim 1 wherein said lipid-regulating agent is a statin.

5. A composition of claim 4 wherein said statin is pravastatin.

6. A composition of claim 4 wherein said statin is atorvastatin.

7. A composition of claim 1 wherein at least one or more of said emulsifiers is selected from phospholipids, polyoxyethylene sorbitan fatty acid derivatives, sorbitan fatty acid derivatives, Polyoxyl-35-castor oil (Cremophor EL, available from BASF), castor oil or hydrogenated castor oil ethoxylates, polyglycerol esters of fatty acids, fatty acid ethoxylates, alcohol ethoxylates, polyoxyethylene-polyoxypropylene copolymers and block co-polymers, and TPGS (d-alpha tocopheryl polyethylene glycol 1000 succinate).

8. A composition of claim 7 wherein at least one or more of said emulsifiers is polyoxyethylene sorbitan fatty acid derivatives, sorbitan fatty acid derivatives and polyoxyl-35-castor oil.

9. A composition of claim 1 wherein said oil is selected from soybean oil, coconut oil, canola oil, corn oil, palm kernel oil, cottonseed oil, olive oil, peanut oil, safflower oil and sesame oil.

10. A composition of claim 9 wherein said oil is soybean oil.

11. A composition of claim 1 further comprising a co-solvent.

12. A composition of claim 11 wherein said co-solvent is ethanol, propylene glycol or polyethylene glycol.

13. A delivery system comprising a composition of claim 1.

14. A delivery system of claim 13 wherein said delivery system is an emulsion.

15. A delivery system of claim 13 wherein said delivery system is a capsule.

16. A method of treating hyperlipidemia comprising the administration of a composition of claim 1 to a patient.

17. A method of treating hyperlipidemia comprising the administration of a composition of claim 3 to a patient.

18. A method of treating hyperlipidemia comprising the administration of a composition of claim 14 to a patient.
